(19) United States
(12) Patent Application Publication
(10) Pub. No.: US 2009/0325171 A1
(43) Pub. Date: Dec. 31, 2009

(54) Vesicles for use in biosensors

(76) Inventors: Thomas Hirt, Rebstein (CH); Zhihua Lu, Johns Creek, GA (US); Janos Voros, Zurich (CH); Dorothea Niederberger, Thalwil (CH)

Correspondence Address: LAW OFFICE OF COLLEN A. BEARD, LLC, P. O. BOX 1064, DECATUR, GA, 30031-1064, US

(21) Appl. No.: 12/454,067

(22) Filed: May 12, 2009

Related U.S. Application Data
(60) Provisional application No. 61/190,832, filed on Sep. 3, 2008.
(60) Provisional application No. 61/198,978, filed on Nov. 12, 2008.

(30) Foreign Application Priority Data
May 13, 2008 (EP) 08008831.3

(51) Int. Cl. C12Q 1/68; G01N 33/53; C40B 60/12

(52) U.S. Cl. 435/6; 435/7.9; 506/39

(57) ABSTRACT

Vesicles for use in biosensors that have both high specificity and high sensitivity, where the vesicles include a receptor specific for the intended analyte and a signal generating component.

### CROSS REFERENCE TO RELATED APPLICATIONS

The present application is related to and claims priority to EP Application No. 08008831.3 filed on May 13, 2008 and to U.S. Provisional Application Ser. No. 61/198,978 filed on Nov. 12, 2008, the entire contents of which are incorporated herein by reference.

### BACKGROUND OF THE INVENTION

The invention relates to biosensors and polymeric vesicles used in biosensors. More specifically, the invention relates to biosensors employing vesicles that provide various means for signal generation and amplification. The biosensors have high specificity and sensitivity.

Biosensors are potentially very useful for early diagnosis of medical conditions, because of their ability to detect biomarkers with high specificity and at very low concentrations. Biomarkers have been identified for many conditions and their detection at early stages in the condition when they are at lower levels could lead to more effective treatment of these conditions. For example, some cancer antigens, such as prostate specific antigen (PSA) and carcinoma antigen (CA-125) have been identified. Eighteen signaling proteins have been identified that indicate whether or not a patient will develop Alzheimer's disease within 2-6 years. Ray et al., Nature Medicine, 13(11), 1359-1362 (2007).

Another application for biosensors is the early and rapid detection of biological toxins, which is critically important for the protection of security personnel deployed in hostile situations or in instances of domestic terrorism. Biological toxins, such as botulinum toxin, are lethal at very low concentrations, which necessitate detection measures that are both highly specific and extremely sensitive. There are a multitude of scenarios that may require the ability to detect biological toxins at sub-attomolar (10⁻¹⁸M) concentrations or even at levels approaching a few molecules. There exists a substantial need for sensors capable of detecting biological toxins, infectious bacteria and viruses, chemical warfare agents, poisons and other chemical toxins, explosive compounds, and trace forensic evidence.

Presently, techniques employed for the selective, sensitive detection of protein antigens include antibody-based immunoassays and DNA-amplification methods. Each of these techniques suffers from drawbacks and problems.

One common bioassay based upon the highly specific interaction between an antigen and antibody is the enzyme linked immunosorbent assay (ELISA). The ELISA has different formats. In one embodiment, an unknown amount of antigen is affixed to a surface, and then a specific antibody is washed over the surface so that it binds to the antigen. This antibody is linked to an enzyme, and in the final step a substrate is added that the enzyme acts upon and in which process some detectable signal is produced. The signal can be quantified and is proportional to the amount of antigen.

Thus in the first step, the antigen of interest is immobilized on a solid support (usually a polystyrene microtiter plate) either non-specifically (via adsorption to the surface) or specifically (via capture by another antibody specific to the same antigen, in a “sandwich” ELISA). Then the immobilized antigen is contacted with the antigen-specific antibody (having the enzyme attached thereto), followed by the enzyme substrate. The substrate is catalyzed into a detectable product.

Generally all enzymatic biosensors function by one of two methods. Either the enzyme converts an undetectable compound of interest into another or series of compounds, which can be detected; or the enzyme is inhibited by the presence of the compound of interest and the enzyme inhibition is measurable and proportional to the amount of the compound of interest. A common enzyme system that is employed as the signal generating component is the glucose oxidase system; wherein glucose oxidase is attached to the secondary antibody. After washing, the substrate glucose is applied and the resulting enzymatic reaction produces electrons which can be measured electrochemically.

ELISAs contain elements common to most biosensors to measure an analyte of interest: 1) a solid support; 2) a receptor specific for the analyte of interest (the antigen-specific antibody); and 3) a signal generating component. In addition, a biosensor must include means for detecting and, preferably, quantifying the signal.

In many cases, the sensitivity of biosensors using a secondary antibody labeled with a signal generating component as described above is insufficient (ELISAs are typically restricted to the nanomolar to femtomolar concentration range) and it would be useful to have an amplification system in addition to the previously listed elements. In many cases, a biomarker, whose detection indicates a particular medical condition, exists at a very low concentration. Even cancer markers with relatively high concentrations, such as PSA, are in the range of a few nanograms per milliliter.

In addition to sensitivity limitations, enzyme based biosensors are often limited in practical application by other factors. For example, the process of immobilizing enzymes uses highly specialized synthesis protocols and is often expensive and time consuming. Moreover, the sensor often requires specialized electrical equipment to be used in conjunction with the immobilized enzyme, such as a pH meter or an oxygen electrode. The shelf-life, thermal stability, and reusability of enzymatic sensors are often problematic for practical application of the technology.

One obstacle preventing a large scale production of enzyme-based sensors is a loss of enzyme activity in even slightly non-biocompatible environments. Conditions to retain enzyme stability include maintaining pH values between 6 and 9, and preventing covalent interactions with the medium.

Immunoassay methods offer outstanding selectivity due to the specificity of the antigen-antibody interaction, but offer only modest sensitivity that is limited in practice to the nanomolar to picomolar concentration range. There are alternatives to using enzymes as the signal generating component. Other signal generating components can be attached to the secondary antibody and detected and quantified colormetrically or via their fluorescence. Some signal generating components directly produce the signal (fluorescence) whereas some act as a catalyst to cause the signal (enzymes and inorganic catalysts).

Methods for signal production using metal nanoparticles as a catalyst instead of enzymes are proposed in U.S. Pat. No. 6,417,340. According to these methods, gold nanoparticles act as a catalyst to reduce silver ions (Ag⁺) to silver (Ag), which is precipitated onto the gold nanoparticles. The silver precipitate functions as another catalyst to allow continuous precipitation of silver around the gold nanoparticles, resulting in an increase in the size of the nanoparticles. The concentrations of biomarkers may be measured with high sensitivity through changes in color (Taton et al., Science, 289, 1757-1760 (2000)), electrical properties (Park et al., Science, 295, 1503-1506 (2002)), and Raman spectrum (Cao et al., Science, 297, 1536-1540 (2002)). The growth of the nanoparticles through the precipitation of the silver is limited to a maximum of 30 nm in this specific method, imposing a lower limit to the sensitivity of these methods.

Some methods for the amplification of signals from biosensors are being explored or are currently in use. The methods rely upon the use of different signal generating components, a greater number of signal generating components, and/or upon the use of different detection methods.

Greater sensitivity can be achieved using amperometric enzyme detection. Enzymes, such as horseradish peroxidase, are linked to the detecting antibody and the product of the enzyme reaction is detected amperometrically through its precipitation on an electrode surface. This technique permits detection of antigen concentration down to the picomolar (10⁻¹² M) level (Alfonta et al., Anal. Chem. 73, 91-102 (2001).

Biochip methods for detecting proteins are a variation of the immunoassay method where antibodies are attached to a membrane in a pattern that can be read by an optical scanner. The signal amplification methods employed are the same as those for other immunoassays and thus the detection limit is at the picomolar level with practical detection limits in the micromolar to nanomolar range. However, the greatest advantage of biochip technology is the ability to screen for up to 20 antigens at one time rather than high sensitivity for any one antigen.

Nanomolar sensitivity has been achieved using single-shell closed-sphere bilayers (liposomes) with diameters of 100 nm containing up to 25,000 fluorophore labeled lipids imbedded in each bilayer. Such liposomes can be covalently linked to antibodies and their fluorescence measured upon binding to the antigen. Since each binding event involves one liposome with multiple signaling molecules, as opposed to a single signaling molecule, high signal amplifications are possible. An issue with the more simple approach of encapsulating fluorophores in the lumen of lipids is the leakage of the fluorescent probes out of the liposomes during storage. Singh et al., Anal. Chem., 72, 6019-6024 (2000).

Another technique providing 10 femtomolar sensitivity involves the use of fluorescence detection based on highly fluorescent Europium chelates as the signal generating component. Heavily labeled Europium chelates (up to 110 total) were covalently linked to streptavidin based conjugates to detect near femtomolar amounts of prostate-specific antigen. Qin et al., Anal. Chem., 73, 1521-1529 (2001).

Another amplification method, polymerase chain reaction (PCR), is used for nucleic acid amplification. A hybrid protein assay, coupling the use of antibodies directed against proteins and PCR and referred to as “immuno-PCR,” has been developed to detect proteins. Immuno-PCR techniques employ one of two approaches for coupling amplification substrates (DNA fragments) to antibodies. Direct covalent attachment of the amplification substrate to the antibody of interest uses the terminal phosphate component of the amplification substrate, or an amplification substrate modified to contain an amine group. Wu et al. Left. in Appl. MicroBiol. 32, 321-325 (2001). Indirect non-covalent attachment of biotinylated amplification substrate and biotinylated antibody to a common streptavidin molecule is described in Sano et al., Science 258, 120-122 (1992) and Niemeyer et al., Anal. Biochem. 246, 140-145 (1997).

In these assays the target protein antigen is immobilized on a support (such as a microtiter plate well) and the antibody-DNA complex is allowed to bind to the immobilized antigen. This is followed by the removal of unbound antibody-DNA complex by extensive rinsing. The bound antigen is then detected and quantified through the PCR amplification of the amplification substrate (DNA) with visualization achieved by gel electrophoresis or a real-time PCR assay. These assays have been employed to achieve detection limits of roughly 6,000,000 to 60,000 molecules.

The immuno-PCR methods described above link a single (or at most four) amplification substrate to each antibody. This severely limits the ability of these methods to detect very low copy numbers of antigens (10-100) as quantification of only a few copies of the target DNA molecule by PCR is often difficult or impossible. Many samples contain Taq polymerase inhibitors that can inhibit or prevent the replication of low numbers of starting DNA molecules. Furthermore, particularly when in the field, contamination of samples with extraneous DNA is a critical concern for samples with low target DNA concentrations. Finally, even where amplification is successful, it entails a large and time consuming number of amplification cycles to produce enough DNA to allow for reliable detection of the amplified product.

In another method, taught in US 2005/0158372, a very low detection limit was achieved by encapsulating 50-1000 nucleic acid amplification substrates within a liposome, binding the liposomes to a target analyte, rupturing the liposomes to release the nucleic acids, and amplifying the nucleic acids by a suitable amplification technique (e.g. PCR). An issue with this technique is that false results may be obtained due to various factors, such as contamination of samples during the PCR or nonspecific binding of the liposomes. Moreover, the use of liposomes presents several issues.

Issues with liposomes include leakage from the liposomes, as mentioned above. Additionally the volume of the hydrophobic compartment available in liposomes to encapsulate a hydrophobic component is very limited. The loading for hydrophilic components is limited due to the negative influence on the stability of liposomes, which results in uncontrolled release. Moreover, liposomes are difficult to handle in terms of manufacture and storage.

Notwithstanding the usefulness of the above mentioned methods, a need still exists for an ideal assay. In view of the various methods described above, it appears that there remains a need for a biosensor combining high specificity with high sensitivity.

### SUMMARY OF THE INVENTION

Vesicles are described for use in biosensors that have both high specificity and high sensitivity. High specificity is provided by the use of highly specific receptors, such as an antibody specific for the particular antigen of interest, and very low nonspecific binding. High sensitivity is provided by use of an effective signal generating component optionally coupled to a signal amplification scheme, and a reduction in nonspecific binding. Vesicles are employed in various embodiments, to carry the signal generating component and optionally the amplification scheme. In a preferred embodiment, the vesicles carry both the signal generating component and the amplification scheme.

### BRIEF DESCRIPTION OF THE FIGURES

FIG. 1 is a schematic illustration of one embodiment of a biosensor using one embodiment of the vesicles of the invention.

FIG. 2 illustrates the adsorption of biotinylated nanoreactors as a function of the nanoreactor concentration to a surface measured using quartz crystal microbalance with dissipation monitoring (QCM-D).

FIG. 3 illustrates the adsorption of biotinylated nanoreactors to a sandwich assay biosensor model at a high antigen concentration, measured as frequency change over time (f) and dissipation over time (D) using QCM-D.

FIG. 4 illustrates the measurement of adsorption of biotinylated nanoreactors to a sandwich assay biosensor model using QCM-D at a low antigen concentrations, as a function of antigen concentration.

FIG. 5 illustrates the frequency change over time in QCM-D for specific versus nonspecific adsorption of vesicles to a surface.

FIG. 6 illustrates signal-to-noise ratio (dissipation) over time of the adsorption of vesicles to a surface with serum as the medium rather than buffer.

FIG. 7 illustrates chronoamperometry detection of enzyme functionalized nanoreactors at concentrations ranging from 4 ug/ml to 200 ug/ml adsorbed to a surface. More vesicles led to a steeper slope (current vs. time). The observed large noise on some of the curves is an artifact of the instrument.

FIG. 8 illustrates chronoamperometry detection with vesicles in a sandwich assay format. The observed large noise in part of the curve is an artifact of the instrument.

### DETAILED DESCRIPTION OF THE INVENTION

As used herein, the following terms have the following definitions.

The term “specificity” refers to how well the bioassay selects the intended analyte and does not incorrectly select unintended compounds.

“Sensitivity” refers to the signal to noise ratio of a signal.

“Amplification” refers to an increase in the signal from one or more signal generating components.

The term “lyse” or “lysing” means that a vesicle is opened in some way to release its contents.

The invention is vesicles for use in biosensors, and the biosensors, that provide both high specificity and high sensitivity. High specificity is provided by the use of highly specific receptors and very low nonspecific binding. High sensitivity is provided by use of an effective signal generating component preferably coupled to a signal amplification scheme and a reduction in nonspecific binding. Vesicles are employed in various embodiments, to carry the signal generating component and/or provide the amplification scheme.

In one embodiment, the biosensor employs a typical ELISA assay in which the secondary antibody is attached to a vesicle. The vesicle also carries one or more signal generating components. The biosensor of this embodiment includes a support to which the antigen of interest is immobilized, preferably via an attached primary specific antibody. Optionally, the support can be exposed to bovine serum albumin (BSA) to reduce nonspecific binding. After binding of the antigen, the biosensor is washed to remove unbound antigen and is then contacted with the vesicles. After binding of the vesicles, the biosensor is again washed to remove unbound vesicles, and then the signal from the signal generating component is detected and measured. In the case where the sensor technique has limited sensitivity to changes in the bulk solution the washing step is not necessary because the signal is generated on the sensor directly.

The vesicles of the invention can be used with different ELISA formats, including the sandwich ELISA, indirect ELISA, and competitive ELISA. In each case, the vesicles have attached thereto a receptor (antibody) that is specific for the antigen, either directly or indirectly.

The biosensor can be supported by any of a variety of solid supports, such as a microtiter plate, glass slide, or polymer support. The solid support is desirably coated with an antibody specific to the antigen, using techniques well known to those skilled in the art. Alternatively, the biosensor can rely upon nonspecific adsorption of the antigen to the support in which case the antibody is not necessary.

In addition to the receptor, the vesicle carries the signal generating component. The signal generating component can be a catalyst, such as an enzyme or a metal that generates the compound that is detectable. The signal generating component could also be an inherently detectable compound, such as a fluorescent compound. The signal generating compound can be attached to the outside of the vesicle, can be encapsulated in the interior of the vesicle, or entrapped in the wall of the vesicle.

Amplification of the signal from the signal generating component can be provided in several ways. In one embodiment, the vesicle carries multiple copies of the signal generating component. In this way a single analyte-receptor binding is amplified by the number of signal generating components carried on or inside the vesicle. In other embodiments, amplification is provided by amplifying the signal from the one or more signal generating components. For example, PCR can be used to amplify the number of copies of a DNA fragment associated in some way with the vesicle. The amplification method could also be a catalyst for another subsequent reaction that is performed after the lysis of the vesicles, or self quenched fluorescence, which generates a large signal after lysis of the vesicles.

Analytes

As used herein, the term “analyte” means a substance or chemical constituent that is to be quantitated in a bioassay. The analyte is typically an antigen, which is a substance that binds with an antibody. Analytes include, but are not limited to, proteins and polysaccharides such as parts (coats, capsules, cell walls, flagella, fimbrae, and toxins) of bacteria, viruses, and other microorganisms. Analytes also include biological and chemical toxins, infectious bacteria and viruses, chemical warfare agents, biological and chemical poisons, food allergans, explosive compounds, and trace forensic evidence. Antigens include PSA, CA-125, and many other cancer antigens, some of which are mentioned in Lin et al. Electrochemical and chemiluminescent immunosensors for tumor markers. Biosens. Bioelectron. 20, 1461-1470 (2005).

Vesicles

As used herein, the term “vesicle” means a hollow particle which may be nano or micro sized. “Vesicle” herein refers to vesicles, nanoreactors, and nanocapsules unless otherwise noted or clear from the context. “Nanoreactors” means vesicles that carry the signal generating component encapsulated in the interior or entrapped in the membrane, wherein the signal generating component (such as an enzyme or inorganic catalyst) is still able to catalyze the intended reaction. This can be achieved, for example, by an appropriate choice of amphiphilic block copolymers that form the membrane and allow the diffusion of the signal generating component substrate and/or by the incorporation of channel forming proteins or synthetic channels. “Nanocapsules” means vesicles where the polymer forming the vesicle membrane is crosslinked to add stability.

Vesicles that can be used in the biosensor include the amphiphilic vesicles described in U.S. Pat. No. 6,916,488 to Meier et al. This patent describes vesicles made from segmented amphiphilic A+B copolymers, where A is hydrophilic and B is hydrophobic, which self-assemble when dispersed in oil or water. In one embodiment, the vesicles are made from an ABA triblock copolymer, where the inner core is hydrophilic, the middle layer is hydrophobic, and the outer shell is hydrophilic. In another embodiment, the vesicles are made from a BAB triblock copolymer. In another embodiment, the vesicles are made from an AB diblock copolymer. The copolymers are formed into vesicles and then optionally polymerized or crosslinked for stability to form nanocapsules. Hydrophilic and hydrophobic segments that can be used are described in U.S. Pat. No. 6,916,488, as well as methods for making vesicles therefrom.

Other types of vesicles can be used, such as the stimulus responsive vesicles in U.S. Pat. No. 6,616,946 to Meier et al. These vesicles are also made with AB or ABA block copolymers but have the additional feature of undergoing a permeability change in response to a stimulus. The vesicles described in the Meier patents can be modified as well to be made from amphiphilic peptides or polymer-peptide conjugates. Other peptide-polymer vesicles prepared by atom transfer radical polymerization are described in Ayres et al., Journal of Polymer Science. Part A. Polymer chemistry, 43(24): 6355-6366 (2005) and Taubert et al., Current Opinion in Chemical Biology, 8(6): 598-603 (December 2004). In a preferred embodiment, the amphiphilic copolymer is a block copolymer having a hydrophobic poly(dimethylsiloxane) middle layer and two water soluble poly(2-methyloxazoline) side blocks (PMOXA-PDMS-PMOXA).

The surface of the vesicles can be designed to minimize nonspecific binding without compromising the stability of the vesicles. For example, nonspecific binding of proteins to the biosensor support is minimized if the outer hydrophilic block is chosen to be polyethylene glycol, polyoxazoline, polyHEMA, or polyvinyl pyrrolidone. Vesicles can have an outer shell that prevents nonspecific binding without compromising their stability, in contrast to stealth liposomes which have been modified so they are not recognized but which makes them less stable.

Advantages of using amphiphilic vesicles include reduced nonspecific binding with proteins and other cellular components, and surfaces in general. In addition, with vesicles, both hydrophilic and hydrophobic molecules can be encapsulated and vesicles have a larger hydrophobic volume compared to liposomes, allowing for encapsulation of a larger number of molecules.

Vesicles are very stable under the conditions likely to be used and allow less leakage of encapsulated molecules, compared to liposomes. The synthetic vesicles are also stable against enzymatic attack which is a major advantage over liposomes and other lipid based systems since the biosensors may be used in biological samples such as blood or wastewater. Stability of vesicles can be further enhanced by using longer hydrophobic segments or by introducing crosslinks in the polymer forming the vesicle membrane to form nanocapsules which will further enhance their life time and shelf life. The chemically crosslinked vesicles are also stable in nonaqueous media such as air, gases, or solvents. Crosslinking of vesicles is described in U.S. Pat. No. 6,916,488 to Meier et al.

The flexibility of the composition of the block copolymers that form the vesicles makes it easy to tailor the mechanical and optical properties of the vesicles (such as viscosity, elasticity, refractive index, or fluorescence) which is important for sensor techniques that are based on such detection principles; and it is also easy to functionalize the surface of vesicles.

Another type of vesicle that can be used are the pH responsive vesicles reported by Du et al. J.A.C.S., 127, 17982 (2005). These vesicles are made out of poly(2-(methacryloyloxy)-ethyl-phosphorylcholine)-co-poly(2-(diisopropylamino)-ethyl methacrylate) diblock copolymers (PMPC-PDPA). The PDPA block is pH sensitive with a pKa value between 5.8-6.6. At physiological pH of 7.4 this diblock copolymer forms vesicles and at pH below 5.5 the vesicles dissociate and release their contents.

As described above with respect to vesicles taught in U.S. Pat. No. 6,616,946 to Meier et al. and vesicles taught by Du et al., the vesicles can be stimulus-responsive, meaning that the permeability of the vesicles can be changed in response to a stimulus. This permeability change can be effected in order to enhance signal generating component movement out of the vesicle, for example. In one embodiment, where the signal generating component is an enzyme, for example, the vesicles can be permeabilized to enable entry of the enzyme substrate into the vesicles and exit of the detectable enzyme product out of the vesicle. In this way the signal can be continuously monitored and the vesicles can be reused. A permeability change can be reversible and does not necessarily lead to lysis of the vesicle.

In another embodiment, the vesicles can be lysed in order to allow exit and detection of the signal generating component or a product it produces. Lysis may also be desirable to allow amplification of the signal. Lysis can be achieved via a permeability change in the vesicle, degradation of the vesicle, or rupturing of the vesicle. Lysis can be achieved through pH changes that affect the solubility and/or polarity of the hydrophobic or hydrophilic block of the block copolymer, and that lead to degradation of one of the segments of the block copolymer. Lysis can also be achieved via the degradation of the linkage between block copolymers or the permeability change of inserted channels. Ways to achieve lysis include pH changes, temperature changes, the addition of surfactants, and through exposure to light, whereas certain bonds undergo a conformation change or certain linkages in the block copolymer are cleaved and the vesicles therefore rupture. Lysis can also be achieved electronically by applying a current or a potential, which will change the physical properties of the vesicle or induce a deformation such that the vesicle ruptures. This can be achieved electrochemically by changing the charge density and/or pH, temperature, or and/or concentration of reactive species close to the surface of the sensor. Such electrochemically induced local changes can then result in the rupturing of the vesicles.

Receptors

As used herein, “receptor” means an analyte binding partner. As discussed above, the receptor can directly or indirectly bind to the analyte and can be directly or indirectly specific for the analyte. By “directly” is meant that the receptor binds to and is specific for the analyte itself; by “indirectly” is meant that the receptor binds to and is specific for an intervening component such as a primary antibody or detection antibody.

A vesicle has attached to it at least one analyte-specific receptor. In one embodiment, this is an antigen-specific antibody. An antibody can be attached to the vesicle using a biotin or neutravidin linker, as taught by the prior art for attachment of antibodies to various entities. Other means of attaching the receptor to the vesicle are via the linker avidin, oligonucleotide, thiol-derivative, nitrilo-triacetic acid containing molecule, oligo-peptide, metal ion, amine, or carboxy-derivative.

Other receptors include, but are not limited to, carbohydrate based ligands that can bind to proteins, toxins, or cell surfaces, oligonucleotides, affibodies, antibody fragments, and zinc fingers.

Signal Generating Components

As used herein, “signal generating component” refers to a component that generates a detectable signal directly or indirectly. Various types of signal generating components have been developed for use in biosensors and can be used here. The signal generating component can be one that is inherently detectable, such as a radioactive, phosphorescent, luminescent, or fluorescent compound. An example is highly fluorescent Europium chelates, which can be covalently linked to streptavidin based conjugates and attached to the vesicles. Qin et al., Anal. Chem., 73, 1521-1529 (2001), can be referred to for general information.

The signal generating component can be one that generates a detectable compound when it catalyzes a reaction upon a substrate, such as an enzyme or inorganic catalyst. Examples include the enzymes glucose oxidase and horseradish peroxidase and inorganic catalysts such as metal nanoparticles. The detectable compound may be detectable via a color change or electrochemically, for example.

The signal generating compound can also be a nucleic acid segment, which is detectable and amplifiable by PCR means.

Other signal generating components rely on changes in weight that can be measured with a microbalance. In one embodiment the change in weight is measured from the vesicle attaching to the analyte, and the vesicle itself is the signal generating component.

Any signal generating component can be used that is adaptable to the analyte being measured, that can be attached to a vesicle, encapsulated within a vesicle, or entrapped within the wall of a vesicle (all referred to as “carried by a vesicle”). Many signal generating components are well known to those skilled in the art and can be readily employed or modified as necessary for use in the vesicles and bioassays described herein. Less well known signal generating components can likely also be used, with necessary modifications.

Detection

The detection method used will depend upon the signal generating component that is employed. Electrochemical detection is fairly well known, and often used with enzymatic signal generating components such as glucose oxidase. Amperometric enzyme detection can be used, as described in Alfonta et al., Anal. Chem. 73, 91-102 (2001). Color changes can also be used to detect enzymatic and catalytic activity of signal generating components. Fluorescence detection can be used for detection and measurement of fluorescing signal generating components such as Europium chelates.

In one embodiment, the vesicles themselves are the signal generating component and accumulation of the vesicles can be detected and measured using mechanical detection with a mechano-sensitive sensor, such as a quartz crystal microbalance (QCM), surface acoustic wave (SAW) sensor, a film bulk acoustic resonator (FBAR), or cantilever resonator. These techniques allow for measurement of in situ changes in mass and viscoelasticity upon binding of vesicles to secondary antigens. An example, as used with antibody modified lipid bilayers, is described in Larsson et al., Anal. Biochem. 345, 72-80 (2005). Due to the low nonspecific adsorption of vesicles to proteins, and vice versa, the detection limit of this method with vesicles should be low. It may also be possible to use these methods to detect the accumulation of products produced by the signal generating component.

In another embodiment accumulation of the vesicles can be measured by the change in refractive index. The refractive index can be measured by surface plasmon resonance, optical waveguide sensor, or ellipsometer. The change in refractive index can either be caused by the solution encapsulated in the vesicles or caused via diffusion of molecules or ions through incorporated channels in the vesicles, e.g. Ca²⁺ through ion channels and the subsequent formation of an insoluble salt. Refractive index can also be used to measure accumulation of the signal generating compound, in cases where the refractive index of the signal generating compound is different from that of the solution.

In another embodiment vesicles can be used with microarrays to analyze DNA or proteins, for example. Techniques making use of labels can be employed, including scanner type, total internal reflection type, fiber optics based, and SPR enhanced fluorescence. State of the art label-free techniques, including imaging SPR and imaging ellipsometry can be used. Combinations of differently labeled vesicles and vesicles with different receptors and/or signal generating components is easily possible, to allow for detection of multiple analytes or detection by different means. Signal amplification schemes as described herein will allow improvement of the sensitivity for microarrays.

Vesicle Design and Amplification Methods

In one embodiment, the signal generating component is attached to the exterior of the vesicle. For example, multiple glucose oxidase molecules can be adsorbed to vesicles as described in Singh et al., Biotechno. Prog 11: 333-341 (1995) or attached to vesicles using biotin or neutravidin linkers. An end group of the polymer used to form the vesicles can be a functional group that can be modified before or after assembly of the polymer into the vesicles so that the enzyme can be attached. Polyethylene glycol and polyoxazolines for example have hydroxyl end groups which can be modified. For polyethylene glycol several other functional endgroups can be created via established processes as can be seen from the many commercially available functionalized oligo ethylene glycols. The endgroups of polyoxazoline can either be modified from the hydroxyl terminus or a secondary amine can be generated by adding an excess of piperazine for the termination of the cationic polymerization.

Catalysts such as transition metals can be complexed onto the vesicle membrane. A chelate for the specific metal ion can be attached to the outer segment of the vesicle forming polymer and the transition metal can then complex with this chelate. Similar to the use of enzymes attached to the exterior of the molecule, this increases the number of catalysts per antibody-antigen binding, amplifying the signal, and allows for a very high sensitivity.

In another embodiment, the signal generating component is encapsulated within the vesicle. Preferably a plurality of signal generating components is encapsulated within the vesicles, allowing for amplification of the signal. For example, a plurality of enzyme molecules or inorganic catalyst molecules can be encapsulated. In one embodiment, a plurality of nucleic acid segments can be encapsulated within a vesicle. After the receptor labeled vesicle is exposed to and bound to the analyte, the vesicle is lysed and the nucleic acid segments are captured, detected, and quantified. PCR can be used to increase the number of copies and even further amplify the signal intensity. U.S. Patent Application 2005/0158372, which describes a bioassay using liposomes encapsulating DNA fragments, can be used for guidance. Immuno-PCR is also described in Niemeyer et al., Anal. Biochem. 246, 140-145 (1997), among other references. Other signal generating components can be encapsulated within the vesicles and quantified within the vesicles (such as in the case of a fluorescent component), or released from the vesicles and quantified. Other amplification methods may be used to even further amplify the signal from these signal generating components or their products.

In embodiments where the signal generating component is encapsulated within the vesicle, the vesicle can be designed to allow for passage of a substrate for the signal generating component into the vesicle and/or product out of the vesicle. This is especially useful where the product is the detectable compound. In one embodiment, the substrate and/or product will simply diffuse through the membrane. In other embodiments, specific channels can be placed in the vesicle wall to enable transfer. Permeability changes can also be exploited to enhance transfer. Alternatively, the vesicle can be lysed to release the signal generating component or a product it generates (detectable compound) which is then detected and quantified.

Channel forming proteins or synthetic channels can be designed into the vesicle membrane. It is possible to insert sufficient channels such that the diffusion processes are not the speed limiting step.

In one embodiment, the opening and/or closing of the channels can be controlled to open or close before or after the binding event of the receptor with the analyte of interest, for example. The opening or closing of the channels can be controlled to allow transport of the substrate or transport of the detectable molecule. The opening or closing of the channels can be controlled by exposure to a stimulus (e.g. an electric field or local pH change), or by other means as further discussed above.

In other embodiments, the vesicle can be lysed e.g. by inducing a local electrochemical change in its vicinity using an electrode, adding surfactants, changing the temperature, changing the pH, or irradiating the sensor with light of a certain wavelength.

One example of a signal generating component that can be encapsulated, and the substrate will migrate into the vesicle, is superoxide dismutase (SOD). There are several known methods for colormetrically detecting SOD activity using reactive oxygen species (ROS) as the substrate. One method is described in Axthelm et al., J Phys Chem B. 112 (28): 8211-8217 (2008).

Advantages of encapsulating the enzyme within the vesicle include the greater stability of the enzyme in the hydrophilic environment inside the vesicle, and the ability to include a greater number of enzyme molecules within the vesicle versus attached to the exterior. In addition, non-specific adsorption caused by the enzyme can be avoided. Since the vesicles protect the enzyme from the environment, the shelf life of the bioassay can be extended.

In another embodiment, the signal generating component can be entrapped in the wall of the vesicle. For example, an enzyme can be entrapped in the vesicle wall, which provides the advantage of not requiring a channel for transport of a substrate or product. Another advantage is that the receptor and the enzyme are spatially and functionally separated from each other. In this embodiment, the active site of the enzyme needs to point to the outside or the substrate to be converted needs to be hydrophobic and penetrate into the membrane. One example of entrapment of an enzyme in a polymer vesicle is described in Winterhalter et al., Talanta 55; 965-971 (2001).

In any of the embodiments described above, more than one type of signal generating component can be employed as well as more than one type of receptor.

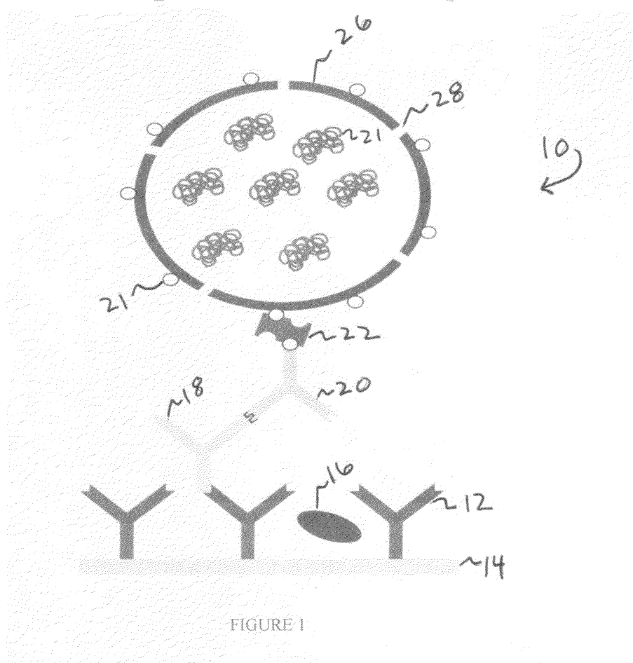

One embodiment of an embodiment of a biosensor 10 employing vesicles of the invention is shown in FIG. 1. The primary antibody 12 is adsorbed to the support 14. Bovine serum albumin 16 is added to prevent unspecific adsorption before the antigen 18 is captured. Subsequently, the secondary antibody 20, coupled to the vesicle via biotin 21 and neutravidin 22, is added. Multiple glucose oxidase molecules 24 are encapsulated within the vesicle 26, which contains channels 28 permeable to glucose, mediator, and electrons.

### EXAMPLES

The examples below serve to further illustrate the invention, to provide those of ordinary skill in the art with a complete disclosure and description of how the compounds, compositions, articles, devices, and/or methods claimed herein are made and evaluated, and are not intended to limit the scope of the invention. In the examples, unless expressly stated otherwise, amounts and percentages are by weight, temperature is in degrees Celsius or is at ambient temperature, and pressure is at or near atmospheric. The examples are not intended to restrict the scope of the invention.

### Example 1

### Synthesis of Biotinylated Amphiphilic Polymer

Further details for the synthesis of amphiphilic polymers can be found in U.S. Pat. No. 6,916,488. An amphiphilic polymer (HO-PMOXA₁₃-PDMS₆₀-PMOXA₁₃-OH, 1.0 g), 200 mg of biotin, and 300 mg of hexamethylenetetramine were added to a 100 ml 2-neck flask and dried under vacuum for 24 h. Then 50 ml of dry trichloromethane was added under nitrogen and the reaction carried out at room temperature for 60 h. Trichloromethane was evaporated under reduced pressure and the polymer was dissolved in an ethanol/water mixture (4/1, v/v). This solution was diafiltrated through a membrane (Mw 1000 cut off) to remove unreacted biotin. The solvent was evaporated under reduced pressure. The polymer was dried under vacuum for 24 h and characterized by ¹H NMR (1.2-1.4 ppm, —CH₂— of biotinyl group). The yield was 50%.

### Example 2

### Formation of Biotinylated Amphiphilic Polymer Nanoreactors

Further details for the formation of nanoreactors from amphiphilic polymers can be found in Nardin, C. et al. Reviews in Molecular Biotechnology 90:17-26 (2002) and Nardin, C. et al. Eur. Phys. J. E 4: 403-410 (2001).

15 mg of HO-PMOXA₇-PDMS₂₂-PMOXA₇-OH and 1.5 mg of the biotinylated polymer of Example 1 were placed in a 10 ml flask and dissolved in 2 ml of ethanol, then 20 μl of a solution of the bacterial porin OmpF (1.5 mg/ml) was added. The solution was vortexed for 1 min and then ethanol was evaporated under reduced pressure. On the top of the film, an additional 10 μl of OmpF solution was placed, and dried under high vacuum.

After film drying for approximately 45 min, 5 ml of glucose oxidase (1 mg/ml, or 200 units/ml) in 100 mM acetate buffer pH 5.5 was added. The film was hydrated under shaking for about 12-15 h at 0 C.

After film hydration, the vesicle solution was filtered through a 1 μm and afterwards at least 5 times through a 400 nm filter (Agilent) with a syringe drive system (Agilent) and placed on a Sepharose-4B column for the separation of the nanoreactors from unencapsulated glucose oxidase and OmpF.

Once prepared, the nanoreactors were kept at 4 C.

### Example 3

### Activity Testing of Nanoreactors in Solution

10 mM glucose in 100 mM acetate pH 5.5 buffer, 100 units/ml horseradish peroxidase (in 100 mM AcH/AcNa pH 5.5 buffer), and 100 uM Amplex-Red were mixed. The solution was colorless to slightly pink depending on the freshness of the Amplex-Red. 50 μl of the nanoreactors of Example 2 were added to the above mixture and the solution turned purple within 1-3 minutes, indicating that the nanoreactors were functional and the glucose oxidase inside the nanoreactors was active.

### Example 4

### Binding of Biotinylated Nanoreactors to a Biosensor Surface

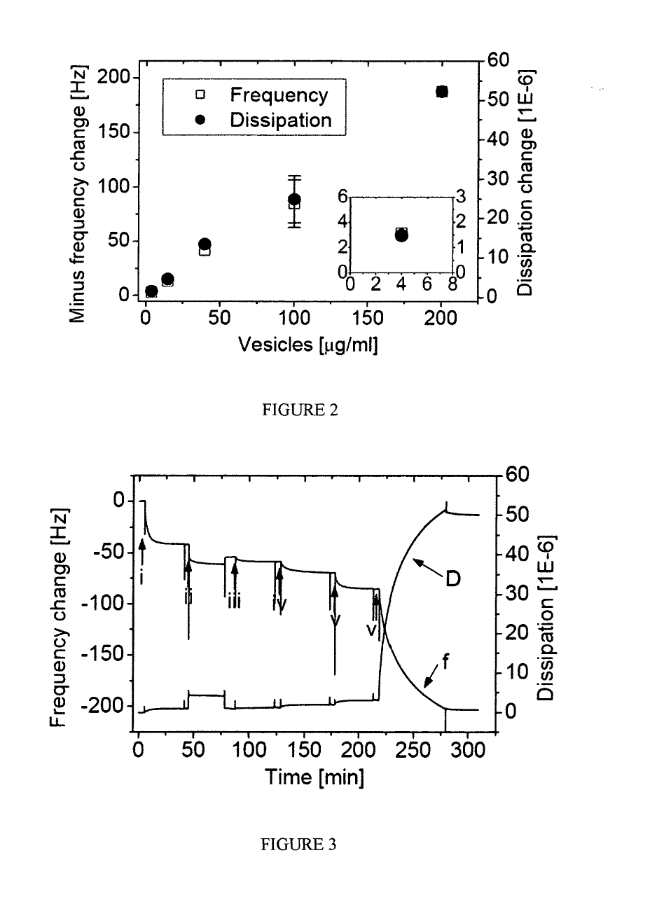

The following example illustrates the application of the system using a support bound model analyte (neutravidin). Neutravidin (20 μg/ml) was adsorbed to a gold surface. Bovine serum albumin (BSA) (0.1 mg/ml) was subsequently adsorbed to block unspecific binding. Then, different concentrations of the biotinylated polymeric nanoreactors of Example 2 were injected. The adsorption was in situ and followed by quartz crystal microbalance with dissipation monitoring (QCM-D). The measurements were performed in 10 mM HEPES buffer, 100 mM KCl, pH 7.4. The results are illustrated in FIG. 2. The graph shows the changes in frequency and dissipation upon adsorption of various concentrations of the vesicles after one hour. At the low concentrations used the sensor readout is linear to the concentration and therefore a further reduction of the detection limit is expected, although pM concentrations are already detectable. (4 ug/ml corresponds to 0.2 pM based on the assumption that the vesicles have a membrane thickness of 10 nm and a polymer density of 1 g/cm³). It is also evident that there is little or no steric hinderance of the vesicles in the measured concentration range.

### Example 5

### Testing of Nanoreactors in a Sandwich Assay Biosensor Model with QCM-D

The following example illustrates the applicability of the nanoreactors in an ELISA format in a biosensor. The results are shown in FIG. 3 and the reference numbers in the following description indicate the reference numbers of FIG. 3. In this example a high antigen concentration was used to illustrate the buildup of each component.

20 μg/ml Fc specific anti-mouse IgG (i) was adsorbed onto a gold surface. Bovine serum albumin (BSA) (10 mg/ml) (ii) was subsequently adsorbed to block non-specific binding. Then, the antigen mouse IgG (2 μg/ml) (iii) was adsorbed, followed by a biotinylated Fab specific anti-mouse IgG (20 μg/ml) (iv), neutravidin (20 μg/ml) (v) and the biotinylated nanoreactors of Example 2 (0.2 mg/ml) (vi). The adsorption was in situ and followed by QCM-D. The measurements were performed in 10 mM HEPES buffer, 100 mM KCl, pH 7.4. Binding of nanoreactors to the support is evident.

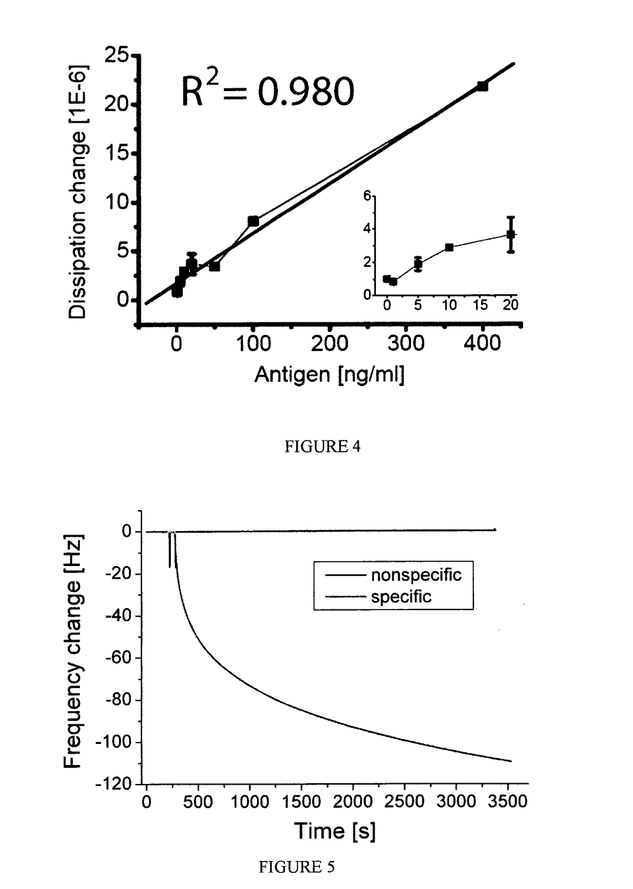

The dissipation change for low concentration of antigen in the sandwich assay is shown in FIG. 4. The same procedure as above was used, with a concentration of antigen from one to 400 ng/ml. The dissipation signal from the QCM-D resulted in a linear signal at this low concentration range, indicating lower detection limits are possible. It also implies that the non-specific adsorption is low and steric hindrance is low.

### Example 6

### Non-Specific Adsorption in Sandwich Assay

QCM-D is an excellent method to test for non-specific adsorption. In this example vesicle adsorption in a sandwich assay with (specific adsorption) and without (non-specific adsorption) antigen present was compared. The protocol of Example 5 was carried out with the biotinylated nonreactors of Example 2. FIG. 5 illustrates that the non-specific adsorption is close to the detection limit of the instrument.

### Example 7

### QCM-D Measurements in Serum

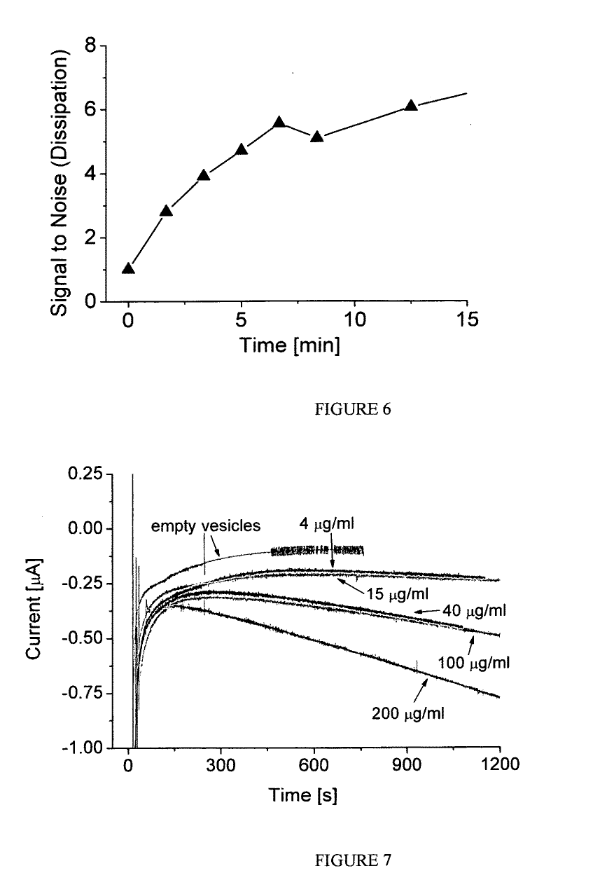

This example illustrates the low signal to noise (S/N) ratio of the vesicles in serum. Experiments were done as in Example 5. The noise values were obtained from nonspecific binding of biotinylated nanoreactor binding. FIG. 6 illustrates the excellent signal to noise ratio and the short response time. Furthermore, serum did not interfere with the measurement, which is essential for many applications.

### Example 8

### Chronoamperometry

This example illustrates electrochemical detection of the biotinylated nanoreactors of Example 2. The biotinylated nanoreactors were bound to the surface as described in Example 4. Then 1 mM ferrocyanide (mediator) and 200 mM glucose (substrate) were added to the buffer. FIG. 7 shows the current developed over time. A potential of 0 V was applied. The amount of antigen was determined by electrochemical detection of the enzymatic activity of the glucose oxidase inside the vesicles. A constant potential around the open circuit potential (OCP) was applied for 10-15 min to obtain the amount of active enzyme. This potential reduced the ferrocyanide ions (previously oxidized through the enzymatic reaction), which allowed for monitoring the amount of enzyme and indirectly the amount of antigen. More vesicles (corresponding to a higher antigen concentration), corresponds to a steeper slope of the readout curve of chronoamperometry with ferrocyanide as a mediator.

### Example 9

### Sandwich Assay

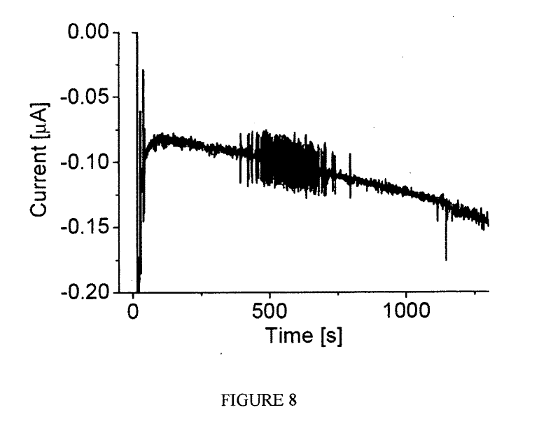

Example 9 is similar to Example 8, except that the nanoreactors were formed into a sandwich assay as described in Example 5. FIG. 8 illustrates the current developed over time for this biosensor.

### Example 10

### Use of a Fluorescent Agent as a Signaling Component

50 mg of PMOXA-PDMS-PMOXA was dissolved in 2 ml of ethanol in a 10 ml flask, and then ethanol was evaporated under reduced pressure. The film was hydrated with 5 ml of 100 mM calcein disodium salt (the fluorescent dye) under stirring for about 96 h. After film hydration, the vesicle solution was filtered through a 0.45 μm filter and then extruded through a 0.22 μm filter with a syringe drive system. The mixture was placed on a Sepharose-4B column for the separation of the vesicles from unencapsulated calcein with 2 mM PBS. The cloudy orange solution was collected. The particle size was 180-240 nm and the yield was 15 ml.

The calcein concentration of the vesicles was determined by absorbance of calcein at 263 nm on a Cary 5 UV-Vis-NIR spectrophotometer against a calcein calibration curve. The concentration of calcein was 2.51 mM.

The fluorescent intensity of 0.5 ml of calcein loaded vesicles in a Costar 4*6 well was measured on a Perkin Elmer 1420 multilabel counter using a lamp filer of F485 and an emission filter of 535. The vesicles were lysed and the fluorescence of the vesicle contents was measured. The counts are summarized in the following table.

| Dilution factor of vesicle solution | 1 | 5 | 10 |
|---|---|---|---|
| Fluorescent counts | 2,353,323 | 1,198,421 | 219,616 |
| Fluorescent counts after lysis | 4,327,968 | 3,070,781 | 544,347 |
| with 100 ul of Triton X-100 (diluted | | | |
| five times) | | | |

The results indicate the fluorescence was detected inside the vesicles. Fluorescence decreased with vesicle dilution, as expected. Since the fluorescent dye can be self-quenching inside the vesicles it was also expected and seen that the fluorescence significantly increased after lysis and release of the dye.

Modifications and variations of the present invention will be apparent to those skilled in the art from the forgoing detailed description. All modifications and variations are intended to be encompassed by the following claims. All publications, patents, and patent applications cited herein are hereby incorporated by reference in their entirety.

What is claimed is:

1. A vesicle for use in a biosensor designed for detection and measurement of an analyte, wherein the vesicle comprises a receptor suitable for binding directly or indirectly to the analyte and a signal generating component that is inherently detectable or can generate a detectable compound.

2. The vesicle of claim 1 further comprising an amplification system.

3. The vesicle of claim 1 wherein the signal generating component is an enzyme or inorganic catalyst that catalyzes the production of a detectable compound, a molecule that is inherently detectable, or a nucleic acid fragment.

4. The vesicle of claim 1, wherein the molecule that is inherently detectable is a molecule that is radioactive, fluorescent, phosphorescent, or luminescent.

5. The vesicle of claim 1, wherein the vesicle has a membrane and the signal generating component is attached to the exterior of the vesicle membrane, encapsulated within the vesicle, or entrapped in the vesicle membrane.

6. The vesicle of claim 1, wherein the vesicle is made from an amphiphilic block copolymer.

7. The vesicle of claim 6, wherein the amphiphilic block copolymer forms the vesicle with an outer shell that provides low non-specific binding.

8. The vesicle of claim 6, wherein the amphiphilic block copolymer comprises polyethylene glycol, polyoxazoline, polyHEMA, or polyvinyl pyrrolidone as the outer hydrophilic block and the vesicle exhibits low non-specific binding.

9. The vesicle of claim 2, wherein the amplification system is a plurality of signal generating components.

10. The vesicle of claim 9, wherein the amplification system is a plurality of signal generating components encapsulated within the vesicle.

11. The vesicle of claim 9, wherein the vesicle has a membrane and the amplification system is a plurality of signal generating components entrapped within the vesicle membrane.

12. The vesicle of claim 9, wherein the amplification system is a plurality of signal generating components on the outside of the vesicle.

13. The vesicle of claim 9, wherein the vesicle can be lysed and the plurality of signal generating components can be detected.

14. The vesicle of claim 9, wherein the plurality of signal generating components is nucleic acid fragments amplifiable by PCR.

15. The vesicle of claim 1, wherein the signal generating compound or the detectable compound has a different refractive index than the solution.

16. The vesicle of claim 1, wherein the signal generating compound is encapsulated by the vesicle and permeability of the vesicle can be changed in order to release the signal generating molecule or detectable compound.

17. The vesicle of claim 16, wherein the vesicle permeability can be changed by changing the temperature or pH, by irradiation, or by the addition of surfactants.

18. The vesicle of claim 1, wherein the vesicle can be lysed to release the signal generating molecule or detectable compound.

19. The vesicle of claim 18, wherein the vesicle can be lysed by changing the temperature or pH, by irradiation, or by the addition of surfactants.

20. The vesicle of claim 16, wherein the vesicle permeability can be changed electronically or electrochemically.

21. The vesicle of claim 18, wherein the vesicle can be lysed electronically or electrochemically.

22. The vesicle of claim 21 where the vesicle can be lysed electronically by applying a current or potential.

23. The vesicle of claim 21 where the vesicle can be lysed electrochemically by changing the charge density or pH, temperature, or concentration of reactive species close to the surface of the sensor.

24. The vesicle of claim 1, wherein the signal generating component is an enzyme that acts on a substrate to make a detectable compound and the vesicle has channels allowing passage of the substrate or mediator into the vesicle or the detectable compound out of the vesicle.

25. The vesicle of claim 24, wherein the channels that can be opened or closed in response to a stimulus.

26. The vesicle of claim 1, wherein the vesicle has a membrane and the signal generating component is an enzyme entrapped in the vesicle membrane.

27. The vesicle of claim 1, wherein the vesicle is made from the amphiphilic block copolymer PMOXA-PDMS-PMOXA and the signal generating compound is glucose oxidase.

28. The vesicle of claim 27, further comprising an amplification system comprising multiple glucose oxidase molecules.

29. The vesicle of claim 6, wherein the amphiphilic block copolymers are crosslinked.

30. A biosensor for detecting and measuring an analyte, comprising a support to which the analyte can be immobilized, a vesicle, and a detection means, wherein the vesicle comprises a receptor suitable for directly or indirectly binding to the analyte and a signal generating component that is inherently detectable or can generate a detectable compound.

31. The biosensor of claim 30, further comprising an amplification system.

32. The biosensor of claim 30, wherein the signal generating component is an enzyme or inorganic catalyst that catalyzes the production of a detectable compound, a molecule that is inherently detectable, or a nucleic acid fragment.

33. The biosensor of claim 30, wherein the vesicle is made from amphiphilic block copolymers.

34. The biosensor of claim 30, wherein the vesicle is made from the amphiphilic block copolymer PMOXA-PDMS-PMOXA and the signal generating compound is glucose oxidase.

35. The biosensor of claim 30, further comprising an amplification system comprising multiple glucose oxidase molecules.

36. The biosensor of claim 30, wherein the biosensor is a microarray.

37. The biosensor of claim 30, wherein the permeability of the vesicle can be changed.

38. The biosensor of claim 30, wherein the vesicle can be lysed in order to release the signaling molecule or detectable compound.
